(19) United States
(12) Patent Application Publication
(10) Pub. No.: US 2026/0166212 A1
(43) Pub. Date: Jun. 18, 2026

(54) Surgical System with Sensor Diagnostic Arrangement and Method Thereof

(71) Applicant: JOHNSON & JOHNSON SURGICAL VISION, INC., Irvine, CA (US)

(72) Inventor: Vadim Gliner, Haifa (IL)

(21) Appl. No.: 18/980,509

(22) Filed: Dec. 13, 2024

(51) Int. Cl. A61M 1/00; A61F 9/007

(52) U.S. Cl. CPC A61M 1/73; A61F 9/00745; A61F 9/00754; A61M 1/774; A61B 2217/005; A61B 2217/007; A61M 2205/3331; A61M 2205/70

(57) ABSTRACT

A surgical system and a respective method are described. The surgical system comprises a handpiece comprising a distal end operable for performing a surgical operation on a patient's eye, a pumping module comprising one or more pumps configured for transmitting and/or evacuating fluids via the handpiece, a pressure sensor assembly comprising one or more pressure sensors positioned at one or more selected positions along a path for fluid flow between the pumping module and the distal end of the handpiece, and configured to provide pressure data indicative of fluid pressure along the path, and a controller comprising one or more processors. The controller is configured and operable to receive the pressure data and operation data indicative of a rotation rate of the one or more pumps, and to process the received pressure data and operation data to determine the operation status of the pressure sensors.

### TECHNOLOGICAL FIELD

The present disclosure relates to surgical systems, and more particularly relates to phacoemulsification systems utilizing one or more pressure sensors.

### BACKGROUND

Phacoemulsification is a surgical procedure used to remove the natural lens tissue from the eye. This procedure may typically be used to treat cataracts, which are associated with clouding and hardening of the eye's lens and can cause blurred vision, difficulty seeing at night, and sensitivity to light. During the procedure, a surgeon makes a small incision in the patient's cornea and inserts the distal end of a probe having a needle to break the cataract tissue into small pieces using ultrasound. The pieces of the lens are aspirated out of the eye and an artificial lens is inserted.

The operation may be performed using a phacoemulsification system comprising a handpiece, which comprises a hollow needle at a distal tip, a vibration mechanism (e.g. one or more piezoelectric crystals), irrigation and aspiration channels, and one or more sensors. The handpiece may be connected via a cable/tubing arrangement to a main unit providing one or more pumps for the aspiration channel, and pumping of irrigation fluid via an irrigation channel. Irrigation and aspiration channels extend through the cable/tubing arrangement to the handpiece, ending at the distal end of the handpiece to provide aspiration of fluids from the eye and providing the irrigation fluid into the eye.

In addition to aspiration of liquids and tissue parts from the eye, the phacoemulsification system operates to irrigate the eye, providing irrigation fluid that maintains intraocular pressure (IOP). Typically, the irrigation and aspiration rates are controlled by an automated process, while irrigation and/or aspiration pressures are monitored to maintain IOP.

### Overview

Monitoring irrigation and/or aspiration pressure during a phacoemulsification procedure is important to maintain IOP, and to avoid damage to the patient's eye. While the phacoemulsification system typically comprises one or more pressure sensors, such pressure sensors may be faulty at times, or malfunction.

Malfunction of the pressure sensors may occur for various reasons, including repetitive autoclaving between procedures, impacts such as falling, manufacturing inaccuracies, or other factors. Such malfunction may compromise the safety of the procedure, affecting an operator/physician's ability to monitor and control IOP. In some cases, the system can continue the procedure, utilizing pressure data obtained from one or more pressure sensors that are still operative. However, to maintain procedure safety, the malfunctioning sensors need to be identified.

The pressure and flow rate of fluid within the irrigation and/or aspiration channels/lines of the system may be associated with operation parameters of one or more pumps providing irrigation fluid or evacuating fluid and tissue material from the surgical site. The present disclosure utilizes pre-stored data indicative of a distribution of pressure and/or flow parameters through the irrigation and/or aspiration channels/lines for a given operation rate of the respective pump. In some examples, the present disclosure utilizes processing to determine the likelihood that a measured distribution of pressure/flow parameters complies with the pre-stored distribution, or indicates a deviation therefrom, which may be associated with a malfunction of one or more sensors.

The present disclosure provides a phacoemulsification system utilizing a pressure sensor assembly, and a method for operating a phacoemulsification system and monitoring operation status of the sensors of the sensor assembly. The technique of the present disclosure utilizes obtaining pressure data using different sensors of the pressure sensor assembly to determine data on the status of the sensors, and to determine if one or more sensors malfunction. In some examples, the technique of the present disclosure directly utilizes data on the variation of the pressure data with respect to the operation rate of the respective pump, to determine the status of the sensors. In some other examples, the technique of the present disclosure utilizes a sensor assembly comprising one or more (in some examples two or more or three or more) pressure sensors along the aspiration and/or irrigation paths, and utilizes pressure variation between the two or more sensors along a common path to determine flow rate of irrigation fluid or aspiration fluid through the path. The technique may thus utilize data on the variation of flow rate with respect to the operation rate of the respective pump to determine the status of the two or more sensors.

### BRIEF DESCRIPTION OF THE DRAWINGS

In order to better understand the subject matter that is disclosed herein and to exemplify how it may be carried out in practice, examples will now be described, by way of non-limiting example only, with reference to the accompanying drawings, in which:

FIG. 1 illustrates a surgical system according to some examples of the present disclosure;

FIG. 2 illustrates a handpiece of a surgical system and a pressure sensor assembly according to some examples of the present disclosure;

FIG. 3 illustrates data in a processing path formed by a pressure sensor assembly and at least one processor according to some examples of the present disclosure;

FIG. 4 exemplifies a method according to some examples of the present disclosure;

FIG. 5 illustrates a method for determining flow rate based on pressure profile according to some examples of the present disclosure;

FIG. 6 illustrates an additional method according to some examples of the present disclosure;

FIGS. 7A to 7E exemplify decision parameters for determining the operation status of sensors according to some examples of the present disclosure.

### DETAILED DESCRIPTION

As indicated above, the present disclosure provides a surgical system, such as a phacoemulsification system. The surgical system includes a sensor assembly for monitoring pressure along one or more fluid paths (also referred to as fluid flow paths) in the system, and is operable for monitoring the operation status of one or more sensors of the sensor assembly. In this connection, an operation status of the one or more sensors may be determined as “operating” or “malfunctioning”, when an operating sensor is considered to provide actual reliable data in accordance with a measured parameter, and a malfunctioning sensor is considered to deviate from the actual measured parameter, and typically requires to be fixed, replaced, or re-calibrated.

In the following description, like elements are identified by the same numeral, and are differentiated, where required, by having a letter attached as a suffix to the numeral.

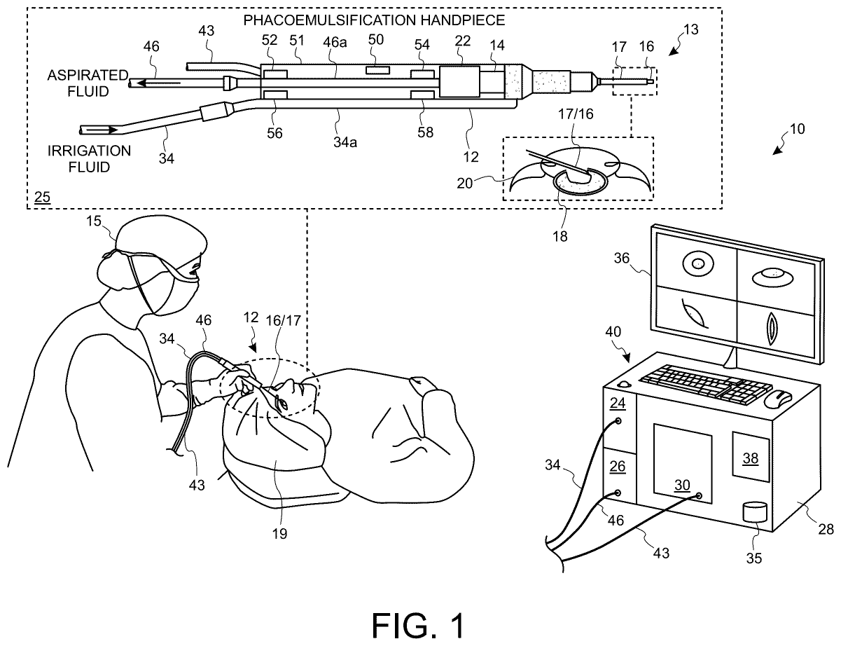

FIG. 1 is a pictorial view exemplifying a phacoemulsification system 10 including a phacoemulsification probe/handpiece 12 and a console 28 connected thereto via one or more cables (such as cables 34, 43, and 46). FIG. 1 includes an inset 25 illustrating additional details of the handpiece 12.

According to some examples of the present disclosure, phacoemulsification probe/handpiece 12 generally includes a hollow needle 16 and a coaxial irrigation sleeve 17. Irrigation sleeve 17 may be positioned to at least partially surround the needle 16, creating a fluid pathway between the external wall of the needle 16 and the internal wall of the sleeve 17. Needle 16 and sleeve 17 are also herein termed a needle-sleeve combination 13. Needle 16 is configured to be inserted into a capsular bag to emulsify lens 18 of an eye 20 of a patient 19. Needle 16 is shown in inset 25 as a straight needle, however, generally, any suitable shape of the needle 16 may be used with the phacoemulsification probe 12, for example, a curved or bent tip needle that is commercially available from Johnson & Johnson Surgical Vision, Inc., Irvine, CA, USA.

The physician 15, operating the system 10 may hold the handpiece 12 so as to perform a phacoemulsification procedure on the eye 20 of a patient 19. The physician 15 may activate the handpiece using a foot pedal (not shown in the figures) or any other suitable user interface, e.g., buttons, voice-activated, etc. Handpiece 12 operates using a vibration generator (e.g., a piezoelectric actuator) 22 configured to vibrate horn 14 and needle 16 connected thereto, in one or more vibration modes of the combined horn and needle. The vibration of needle 16 is used to break up natural lens 18 into small pieces during the phacoemulsification procedure.

Phacoemulsification system 10 utilizes at least one controller 38 typically including at least one processor and corresponding memory units 35, typically associated with console 28. The at least one controller 38 is configured to execute selected computer readable codes, including instructions for operating the phacoemulsification system 10 in one or more operation modes and in accordance with techniques described herein. It should be noted that certain actions associated with operation of the phacoemulsification system 10 are known in the art and are not specifically described herein. The operation of the at least one controller 38 include software and/or hardware functional modules relating to actions described in more detail below. One or more of the functions of the one or more processors of controller 38 may be carried out by suitable software stored in a memory 35. The software may be downloaded to a device in electronic format, over a network, for example. Alternatively, or additionally, the software may be stored in tangible, non-transitory computer-readable storage media, such as optical, magnetic, or electronic memory. Some or all of the functions of controller 38 may be combined in a single physical component, or, alternatively, implemented using multiple physical components. The physical components may comprise hard-wired or programmable devices, or a combination of the two.

Vibration generator 22 of the handpiece 12 is powered by a driving module 30 that may be placed within console 28. Module 30, under overall control of the controller 38, is configured to provide power to the vibration generator 22, via a microcontroller 50, e.g., located in the handpiece 12. Power for microcontroller 50, as well as control signals for the microcontroller, may be delivered to the microcontroller by a cable 43 from driving module 30.

During the phacoemulsification procedure, an irrigation pump 24, which may be located within console 28 or externally thereto, operates to pump irrigation fluid through an irrigation channel 34a in handpiece 12 and toward irrigation sleeve 17, so as to irrigate the eye. The fluid is pumped via an irrigation tubing line 34, running from the pump, which is coupled with channel 34a of the probe 12.

Additionally, an aspiration pump 26, which also may also be located within console 28 or externally thereto, operates to aspirate aspiration fluid, comprising fluid and waste matter (e.g., emulsified parts of the lens), from the patient's eye 20 via needle 16, through an aspiration channel 46a in handpiece 12. Aspiration pump 26 produces a vacuum and is coupled with the pump to aspiration channel 46a by an aspiration tubing line 46.

The irrigation pump 24 and the aspiration pump 26 may be any pump known in the art (e.g., a peristaltic pump, a progressive cavity pump, or Venturi pump), and the pumps are under overall control of the controller 38. The irrigation and aspiration pumps operate to aspirate fluids from the patient's eye, and irrigate the patient's eye with irrigation fluid, to thereby maintain IOP and chamber stability. Generally, operation of the irrigation 24 and aspiration 26 pumps is controlled to maintain a desired pressure range within the patient's eye, to avoid damage to the structure of the eye.

To provide monitoring of pressure, the phacoemulsification system 10, and specifically the handpiece thereof, includes a pressure sensor assembly 51 including one or more sensors. The pressure sensor assembly 51 generally includes a selected number of sensors positioned for monitoring pressure variations along the aspiration and/or irrigation paths. In the exemplary configuration illustrated in FIG. 1, a first aspiration pressure sensor 52, also herein termed aspiration pressure sensor A, is coupled with aspiration channel 46a, in a proximal section of the channel, so as to couple with the aspiration fluid in the channel. A second aspiration pressure sensor 54, also herein termed aspiration pressure sensor B, is also coupled with aspiration channel 46a, in a distal section of the channel, so as to couple with the aspiration fluid in the channel. According to some examples of the present disclosure, the pressure sensor assembly 51 may include at least one additional aspiration sensor, positioned in the vicinity of aspiration sensor A, aspiration sensor B, or at a separate location. The aspiration pressure sensors are located at a known distance between each other, measured along aspiration channel 46a. Pressure variation between the sensors can thus be described in accordance with Bernoulli's principle of hydrodynamics.

Similarly, the pressure sensor assembly 51 may include one or more irrigation sensors including a first irrigation pressure sensor 56, also herein termed irrigation pressure sensor A, coupled with irrigation channel 34a in a proximal section of the channel, and a second irrigation pressure sensor 58, herein termed irrigation pressure sensor B, also coupled with irrigation channel 34a, in a distal section of the channel. Further, as described above, the irrigation sensors may include at least one additional sensor located in the vicinity of irrigation pressure sensor A and/or of irrigation pressure sensor B. The irrigation pressure sensors are located at a known distance between them, the distance being measured along irrigation channel 34a, providing that pressure variation between the sensors can be described in accordance with Bernoulli's principle of hydrodynamics.

The pressure sensor assembly 51, and the different aspiration and/or irrigation pressure sensors thereof, provide pressure output data to the at least one controller 38, using one or more communication lines and/or using wireless communication. For example, signals generated by the aspiration sensors, e.g., sensor 52 and sensor 54, and by the irrigation sensors, e.g., sensor 56 and sensor 58, are provided via cable 43, as illustrated in FIG. 1, to controller 38. In some additional examples, the sensor arrangement 51 may include additional irrigation and/or aspiration sensors. Thus, in some examples the sensor arrangement may include two or more sensors along the irrigation and/or aspiration path (including the irrigation/aspiration line and/or the irrigation/aspiration channel) Typically, the one or more pressure sensors operate to provide pressure data at a selected sampling rate, e.g., providing pressure data at a sampling rate of 1 KHz, 100 Hz, or any other selected sampling rate. The at least one controller 38 uses the pressure data collected from the pressure sensor assembly 51 to determine suitable aspiration and irrigation flow rates, and to provide suitable indication signals to the operator. Additionally, according to some examples of the present disclosure, the controller utilizes the pressure data, in accordance with operation data of the irrigation pump 24 or aspiration pump 26 to determine operational status of the sensors.

Typically, the irrigation or aspiration flow rate may be determined in accordance with rotation rate of the respective irrigation pump 24 and/or aspiration pump 26, and/or using one or more flow sensors. In some examples as described further below, the flow rate may be determined in accordance with a correlation between pressure variation collected by two or more pressure sensors at different locations the along flow path.

The apparatus illustrated in FIG. 1 may include various additional elements, which are omitted for clarity of presentation. For example, physician 15 typically performs the procedure using a stereomicroscope or magnifying glasses, neither of which are shown. Physician 15 may use other surgical tools, in addition to probe 12, which are also not shown to maintain clarity and simplicity.

According to some examples of the present disclosure, the at least one controller 38 is configured and operable for monitoring the operation status of the different sensors of the sensor assembly 51. Monitoring of the sensors'operation status may be performed continuously, periodically or in response to one or more signals. For example, a monitoring operation may be acting in the background during operation time of the system. Alternatively, a monitoring operation may be performed within time cycles of seconds or minutes, to maintain processing power. The controller 38 may provide an indication of operation status of the sensor assembly 51 and the different sensors thereof, and may provide an alert to the physician if a number of malfunctioning sensors exceeds a predetermined threshold, e.g., providing an alert for each malfunctioning sensor, generate an alert when two sensors malfunction, or when more than two sensors malfunction. Further, the controller 38 may store sensor operation status data, and additional data on the operation of the sensors in a memory, and/or transmit such data to a remote storage/processing for further analysis.

Further, the at least one controller 38 may perform additional processing on the pressure data, including one or more additional data, such as rotation rate of the aspiration and/or irrigation pumps, vibration mode and frequency of the vibration generator 22, etc. The at least one controller 38 may generate output data indicative of the processing, and provide output data through a corresponding user interface 40, e.g., using display 36. In an example, user interface 40 and display 36 may be one and the same, such as a touch screen graphical user interface.

The at least one controller 38 may receive user-based commands via a system user interface 40. Such user-based commands may include, but are not limited to, setting and/or adjusting a vibration mode and/or a frequency of the vibration generator 22, setting and/or adjusting a stroke amplitude of needle 16, and setting and/or adjusting an irrigation flow rate and an aspiration flow rate of irrigation pump 24 and aspiration pump 26.

While physician 15 may set the irrigation flow rate and the aspiration flow rate to selected values, or may set the rates to respective default values, it will be understood that the actual rates of flow of both the irrigation fluid and the aspiration fluid may vary with time. The pumps themselves do not deliver the fluid at a constant rate, but rather vary the rate about a nominal set value in an oscillatory manner. In addition, flexing of the tubing connected to the pumps, as well as changes in the tubing terminations at the eye due to the procedure being performed, may cause the flow rate to change.

Generally, during a phacoemulsification procedure, irrigation fluid may flow through the irrigation channel, and aspiration fluid may flow through the aspiration channel. In the case of an occlusion, the fluid flow may stop for certain period. The pressure registered by the aspiration and irrigation sensors, may vary from the IOP due to flow in the channel. In some examples, using the irrigation fluid pressure, IOP can be estimated based on the pressure in the irrigation channel. Accordingly, pressure data collected along the irrigation and aspiration channels can provide an operator/physician with valuable data on operation of the system as well as enable estimation on the patient's IOP. Thus, while not being directly equal, the pressure in the irrigation and/or aspiration channels provides an indication of the IOP, as well as an indication on a variation in the flow rate of the fluid being transferred. Accordingly, monitoring pressure data provides an important input to the physician 15 for safely performing the procedure.

While the phacoemulsification system 10 may preferably be manufactured of high-grade materials using high-quality elements, the different pressure sensors may malfunction for various reasons. To enable monitoring of the pressure data and detection of malfunctioning sensors, the phacoemulsification system of the present disclosure utilizes a pressure sensor assembly 51 including one or more sensors along at least one of the irrigation channel 34a and aspiration channel 46a. Additionally, the at least one controller 38 is configured and operable for analyzing pressure data of the one or more sensors to determine if one or more of the sensors may be malfunctioning.

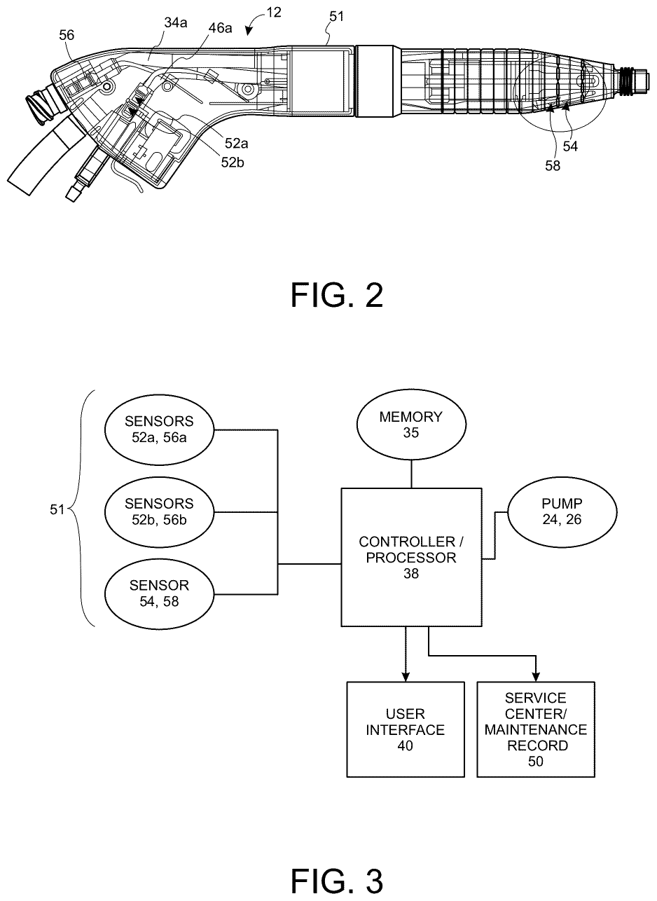

In this connection, reference is made to FIG. 2 exemplifying a probe/handpiece unit 12 of a phacoemulsification system according to some examples of the present disclosure. The handpiece 12 is generally configured as described above with reference to FIG. 1, and specifically illustrates a sensor assembly 51 exemplified herein as including sensors 52a, 52b and 54 along the aspiration channel 46a, and sensors 56 and 58 along the irrigation channel 34a as shown in FIG. 2. Generally, however, the number of sensors along each one of the irrigation channel 34a and aspiration channel 46a may vary in accordance to system and handpiece design, providing one or more sensors along each of the channels.

The pressure sensors of sensor assembly 51, exemplified by pressure sensors 52a, 52b, 54, 56, and 58, collect pressure data indicative of pressure within the irrigation channel 34a or the aspiration channel 46a at a selected sampling rate, and transmit the pressure data to the at least one controller 38. Given a distance between the proximal sensors 52a, 52b, and 56, and the distal sensor 54 and 58, it is expected that these sensors will have certain pressure variations in accordance with Bernoulli's principle of hydrodynamics. Additionally, the temporal profile of the pressure variation is delayed in accordance with fluid flow rate through the channel. In some examples as described in more details below, the controller may utilize the different locations of the present sensors to determine flow rate parameters through the channels.

More specifically, as indicated above, the irrigation pump 24 and the aspiration pump 26 typically provide a pulsed profile of pump operation. As a result, fluid flow may show certain pressure spikes through the irrigation channel 34a and/or the aspiration channel 46a, and the pressure spikes propagate in accordance with the flow rate through the channels. Accordingly, when irrigation fluid is pushed through the irrigation channel 34a, a pressure spike may be first detected by the proximal sensor 56, and after a certain period associated with the distance between the sensors and flow rate through the channel, detected by the distal pressure sensor 58. This is similar to pumping fluid through the aspiration channel 46a, where the aspiration pump 26 operates to pull/evacuate fluid through the channel. Further, each pressure spike may be characterized by a maximal pressure, being the peak of the pressure spike, a minimal pressure, being the shallow between pressure spikes, and a pressure variation being the difference between maximal pressure and minimal pressure.

An additional result of the pulsed operation profile of the irrigation pump 24 or the aspiration pump 26 relates to variations in the flow rate of the fluid flowing through the irrigation channel 34a or the aspiration channel 46a. More specifically, the pulsed operation of the pump results in a cyclic flow rate including a maximal flow rate associated with a pressure spike, followed by a minimal flow rate associated with a reduced pressure following a pressure spike. A typical operation of the irrigation pump 24 or the aspiration pump 26, given predetermined characteristics of the fluid flow path, including irrigation or aspiration line and irrigation or aspiration channel, provides an expected behavior of the flow rate variations. For example, the farther the sensors from the pump, the flow rate variations may show a shallower profile, sensors that are closer to the respective pump may measure a sharper pressure and flow rate variations.

Accordingly, as indicated above, the one or more pressure sensors operate to send pressure data collected using a selected sampling rate, where each pressure data piece may be associated with a time indicator. The pressure data is transmitted to the controller 38 for processing, determining pressure and/or flow parameters and determining operation status of the pressure sensors. The processing is typically performed in accordance with pump operation data, indicative of pump rotation rate, and pre-stored data stored in the one or more memory units 35.

Further, reference is made to FIG. 3 exemplifying a signal communication path between the pressure sensor assembly and the at least one controller 38 according to some examples of the present disclosure. As shown, the different sensors of the sensor assembly 51, including e.g., sensors 52a, 52b, 54, 56a, 56b, and 58, are configured to transmit, via communication wires or by wireless communication, sensing data including pressure data to the controller 38. The controller 38 may further obtain pump rate data (e.g., revolutions per minute) from the irrigation pump 24 and/or aspiration pump 26, and may generally obtain pre-stored data from the one or more memory units 35. The controller 38, and/or one or more processors thereof, operates for processing the received data and determining one or more correlations between the measured data collected by the sensors and the operation data of the pumps with respect to pre-stored data, to determine data on the operation status of the sensors, e.g., sensors 52a, 52b, 54, 56a, 56b, and 58. As indicated above, operation status may be determined as operating or as malfunctioning. Generally, the controller may be configured and operable for receiving the pressure data and operation data indicative of a rotation rate of the one or more pumps (24, 26), for processing the received pressure data and operation data, and determining the operation status of the one or more pressure sensors.

In this connection, the pre-stored data generally includes data on the correlation between pressure variation along the irrigation channel and/or aspiration channel and the operation rate of the respective pump. Further, in some examples, the pre-stored data may include data on the correlation between the flow rate of fluid through the irrigation channel and/or aspiration channel and the operation rate of the respective pump. In some additional examples the pre-stored data may include data on ratio of pressure measurements collected by two different sensors along a common path with respect to the operation rate of the respective pump. Such correlation data may be in the form of a distribution or number of instances of different pressure values or flow rate values associated with the respective operation rate of the pump, and may be presented in the form of a histogram or probability distribution, as indicated in more detail further below.

As indicated above, controller 38 generally includes one or more processors and is in data communication with the one or more memory units 35. The one or more processors are operable for executing computer-readable instructions, generally stored in the one or more memory units 35 and carrying computer instructions for operation of the surgical/phacoemulsification system in accordance with one or more selected operation schemes, and to perform sensor analysis in accordance with the technique described herein. The one or more processors may operate in accordance with computer readable data stored in the memory 35, and may operate to store temporary and other processing data in the memory 35 during operation.

After obtaining pressure data from the sensors (52a, 52b, 54, 56a, 56b, and 58), the controller 38 operates to determine one or more pressure and/or flow parameters based on the pressure data, and determine a relationship between the one or more pressure and/or flow parameters and data on rotation rate of the respective irrigation pump 24 or aspiration pump 26. The controller further utilizes the pre-stored data to determine a likelihood that the relationship between pressure and/or flow parameters and rotation rate of the pump, fits into expected probability in accordance with the pre-stored data. In some examples, the pressure parameter may include average pressure reading of the one or more sensors. In some examples, the pressure parameter may include a ratio between pressure reading of two sensors along a common one of the irrigation or aspiration channel. Further, in some examples, the controller may operate to determine flow rate in accordance with temporal correlation between pressure reading of two or more sensors along a common path, and determine a correlation between the flow rate parameter and rotation rate of the respective pump.

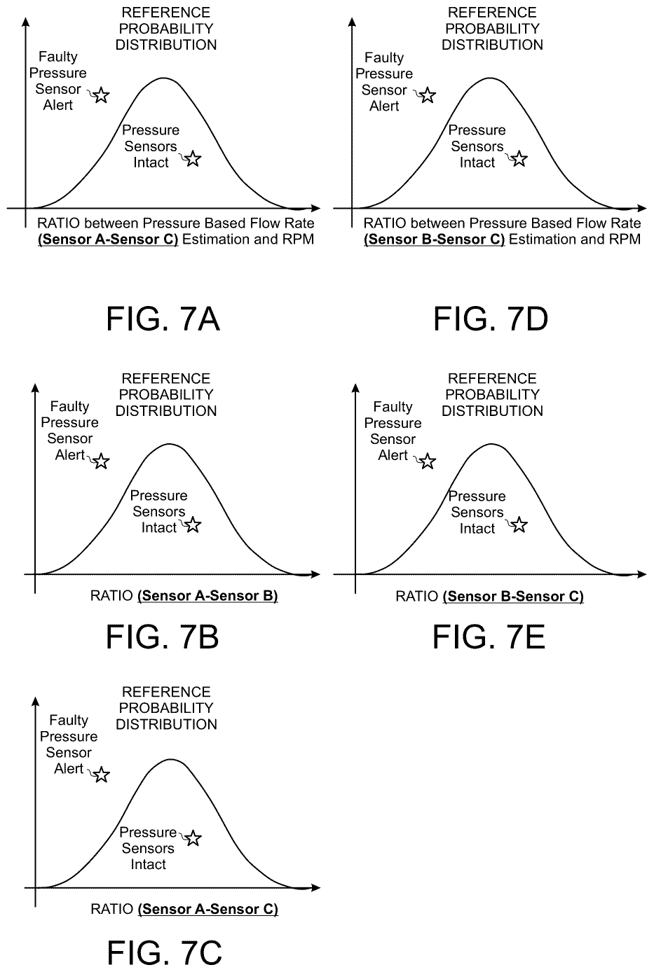

Generally, controller 38 may utilize the pre-stored data to determine a likelihood that the obtained sensing data is within a pre-calculated distribution. This helps the controller 38 in determining whether the readings obtained from one or more sensors is “normal” or if the reading is “abnormal”. Abnormal readings may indicate a malfunctioning sensor. This is exemplified and described in more detail with reference to FIGS. 7A to 7E below. Typically, a well-operating sensor provides sensing data that is reproducible and relates to the actual pressure within the respective irrigation or aspiration channel. Such actual pressure data is expected to correlate with the rotation ate of the respective pump, in accordance with the pre-stored data, generally describing a probability distribution of the correlation. Accordingly, if the determined correlation between the pressure and/or flow data and the rotation rate of the pump does not fit the pre-stored probability distribution, the controller 38 may determine that one or more of the sensors providing that sensing data may be malfunctioning.

After determining the operation status of the one or more pressure sensors, the at least one controller 38 may be further configured to generate an output signal and transmit the output signal to the operator/physician via user interface 40. The output signal may be visual, tactile, and/or audible. Additionally, in some examples the at least one processor may transmit operation status data to be stored in maintenance records or sent to a service center 50. The maintenance records 50 may be used for recording required maintenance actions such as replacing one or more malfunctioning sensors, calibrating sensors, etc., and may further be used to determine required operations by a service center for ongoing maintenance of the system.

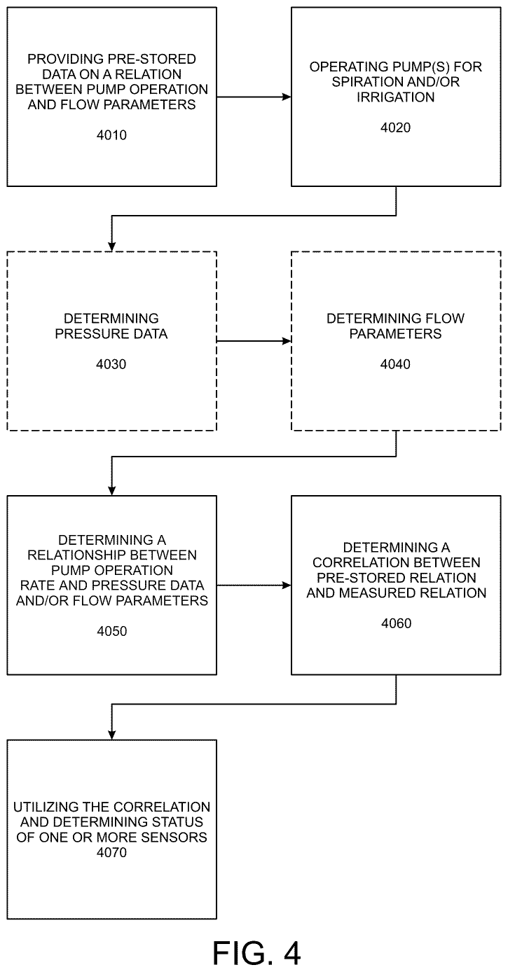

Reference is made to FIG. 4 exemplifying a method for monitoring sensor operation status according to some examples of the present disclosure. The method includes providing pre-stored data indicative of a relationship between pump operation rate and one or more flow parameters (4010). As indicated above, the one or more flow parameters may relate to pressure variation, flow rate, as well as one or more parameters of flow rate, such as flow rate variation (e.g., a difference between maximal flow rate and minimal flow rate). The pre-stored data may be provided as pre-stored in a memory unit of the surgical system, or accessible via a network connection. In some examples, the pre-stored data may be continuously updated using data collected during operation of the surgical system. Generally, providing the pre-stored data may be an initial action, associated with obtaining access to the surgical system.

Further, the method includes operating the one or more pumps of the surgical system for transmitting/evacuating irrigation and/or aspiration fluid through the respective channels (4020). The one or more pumps may be operated at a selected operation rate, determined in accordance with one or more surgical parameters, operation preferences of the operator, etc. During operation of the pumps, and any action performed by an operator/physician, the method may include at least one of determining pressure data of fluid transmitted by the pump (4030) and determining flow parameters (4040). For a phacoemulsification system, the pump may include either one of the irrigation and aspiration pumps, and determining pressure data generally relates to the respective one of the irrigation and aspiration channels and/or lines. Generally, in some examples, both pumps operate with selected irrigation and aspiration operation rates, and pressure data is collected along both the irrigation and aspiration channels. As indicated, the method may include one or more of determining pressure data (4030) and determining flow parameters (4040) based on the temporal correlation of sensing data from two or more sensors. In this connection, the pressure data may include data on pressure variation, ratio between pressure reading of two or more sensors, etc. The flow rate data may be determined, as described above, in accordance with temporal correlation between sensing data provided by two or more sensors along a common path. Such flow parameters may include flow rate, variation in flow rate, difference between maximal flow and minimal flow, etc. The pressure data may include data on pressure variation parameters such as the difference between minimal pressure and maximal pressure, pressure variation frequency, etc.

Based on one or more of the pressure data and the flow parameters determined in accordance with sensing data obtained from the sensors, the method includes determining a relationship between the pump operation rate and the pressure data or flow parameters (4050). Such relationship generally relates to a plurality of measured instances, typically associated with the sampling rate of the sensor assembly. In this connection, the relationship may be formed based on a plurality of measurement pairs, where each measurement pair includes a first data piece indicative of pump rate, and a second data piece indicative of a measured pressure or a parameter relating to fluid flow determined based on sensing data collected at the same time, forming received pressure/flow data and operation data pairs. The plurality of data pairs can thus be arranged as a probability distribution, or histogram, indicating the number of occurrences of each measured value of pressure data or of the flow parameter data, for a respective operation rate of the pump. Accordingly, the determined relationship may represent a distribution of pressure data or flow parameter determined and the respective operation rate of the pump.

The method further utilizes the pre-stored data, which includes a previously collected relationship between the pressure data/flow parameters and operation rate of the pump, and operates to determine a correlation between the pre-stored relationship and the received relationship between the received pressure data/flow parameters and the operation data/rate (4060). The method utilizes the correlation and determines the operation status of the one or more sensors for the sensor assembly (4070), and may generate an output signal indicating if the sensors are operational or malfunctioning.

Determining a correlation between the stored relationship and the received relationship may be performed as a statistical comparison between distributions. Generally, the method may utilize processing of the received and stored relationship to determine a likelihood that the received and stored relationship indicate similar statistical behavior of the pressure data and/or flow parameters with respect to the operation rate of the pump.

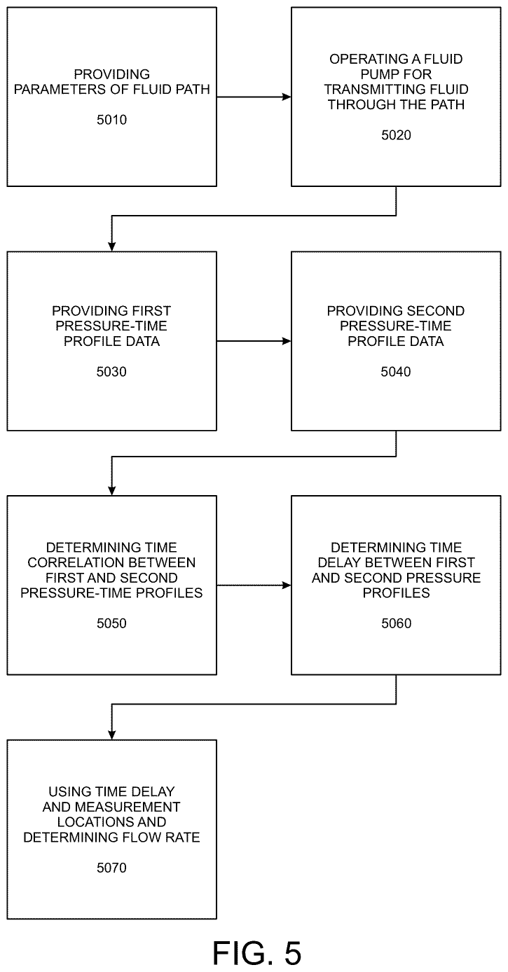

As indicated above, the present disclosure may utilize pressure data or one or more derivatives thereof, such as pressure variation between minimal and maximal pressure, typically associated with pulsed operation of the irrigation and/or aspiration pump. Additionally, or alternatively, the technique of the present disclosure may utilize the pressure data for determining one or more flow parameters indicative of fluid flow through the respective channel. Reference is made to FIG. 5 exemplifying a method for determining flow parameters based on pressure data. The method includes providing parameters of fluid flow path (5010), which may include data on a channel length between first and second pressure sensor locations, channel width/diameter, etc. Additionally, the method includes operating a fluid pump for transmitting fluid through the path (5020), and providing first pressure-time profile data (5030) and second pressure-time profile data (5040). The pressure-time profile data may be a series of data pieces, each indicating a pressure measurement collected by a sensor at a selected location along the path at respective times (in accordance with the sampling rate of the sensor). Accordingly, the pressure-time profile is indicative of pressure variation along the measurement time. The first pressure-time profile is collected at a first sensor location, and the second pressure-time profile is collected at a second sensor location. Determining the time correlation between the first and second pressure-time profiles (5050) may be associated with aligning the first and second pressure-time profiles and determining a time delay (5060) between them. More specifically, the correlation between the first and second pressure-time profiles may be a time delay correlation, indicative of a time difference that provides a high correlation between the profiles. Upon determining a time delay between the pressure-time profile determined at the location of the first sensor, and the pressure-time profile determined at the location of the second sensor, the method uses the time delay and measurement locations (i.e. locations of the respective sensors) for determining flow rate (5070) of fluid through the respective path. As described above, the flow rate may be determined based on temporal correlations of sensing data collected at two different locations along path of fluid flow, and the fluid flow path parameters.

As indicated above, the technique of the present disclosure may utilize pressure data or parameters thereof and/or flow parameters thereof for determining the relationship between measured data and the operational rate of the pump. Typically, pressure variation may be associated with flow variation through the channel, enabling the present technique to operate in accordance with the parameters that are more suitable for the surgical operation.

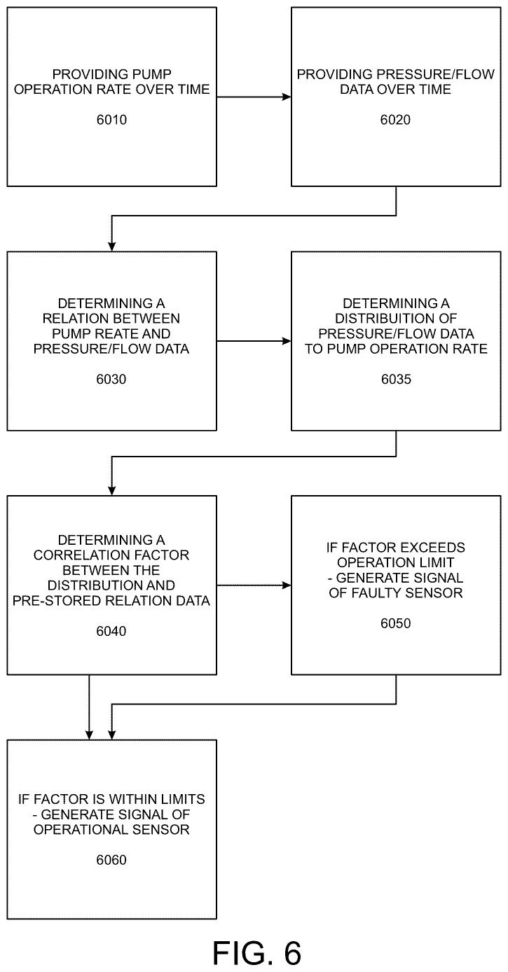

Reference is now made to FIG. 6 exemplifying a method according to some additional examples of the present disclosure. As shown in FIG. 6, the method includes providing pump operation rate (6010) and providing data indicative of the pressure data and/or flow parameters over operation time of the system (6020). Both data types may be determined over time in accordance with sensing data collected by the sensors at a selected sampling rate. The flow parameters may be determined as indicated above using two or more pressure sensors located at two or more different locations along the fluid flow path.

The method further includes determining a relationship between the pump operation data and pressure data/flow parameter (6030). In this connection it should be noted that the term “pressure/flow data” generally relates to pressure data and/or flow parameters described throughout the present disclosure. As described above, the relationship may be a statistical relationship, distribution of values, and/or histogram between the pressure/flow data and the pump operation rate (6035). The method utilizes the relationship and pre-stored data on the relationship between the pump operation rate and pressure/flow data and determines a correlation factor between the received distribution and the pre-stored data (6040). The correlation factor is indicative of how close the received distribution is to the pre-stored data, and indicates a likelihood that an equal or similar distribution describes the two relationships. If the factor exceeds a predetermined operation limit, the method concludes that one or more of the sensors is faulty (6050) and generates a corresponding signal. If the factor is within the predetermined limits (6060), the method concludes that the sensors are operative, and may generate a corresponding signal. Generally, the correlation factor may be determined based on a plurality of data points, collected along a selected period. Accordingly, the correlation factor may be determined by analyzing the plurality of data points and determining if the correlation between the measured/determined data and the pump operation rate is aligned with the pre-stored data, i.e., distribution of the plurality of data points with respect to operation ate of the pump fits the pre-stored data on the distribution/histogram, the relevant sensors are labeled as operating.

Generally, in some examples, the method may further include updating the pre-stored data with newly collected data on the relationship between the pressure data and/or flow parameters and operation rate of the pump. Such newly collected data may include data indicative of the determined operation status of the one or more sensors. For example, during operation, newly collected relationship data may be added to the pre-stored data with an indication that the sensors are considered to be operating properly. Alternatively, if one or more sensors are considered malfunctioning, the newly collected data may be stored with an indication that one or more sensors are malfunctioning. Accordingly, the pre-stored data may include two data sets, a primary data set including relationship data associated with properly operating sensors, and a secondary data set including relationship data associated with one or more malfunctioning sensors. This may simplify processing and enhance accuracy in determining the operation status of the one or more sensors.

Reference is further made to FIGS. 7A to 7E exemplifying relationship data indicative of proper operation or malfunction of one or more sensors. FIGS. 7A and 7B exemplify relationships between flow rate as determined by correlation between two sensors (A and C, and B and C, respectively) aligned along the common fluid flow path, being the irrigation channel or aspiration channel, and rotation rate of the respective pump (RPM). FIGS. 7C to 7E exemplify decision-making, based on the ratio between pressure data collected by different sensors for the respective rotation rate of the pump. FIG. 7C utilizes a ratio between sensor A and sensor C, FIG. 7D utilizes a ratio between sensor B and sensor C, and FIG. 7E utilizes a ration between sensor B and sensor C. Generally, in some further examples, the decision on operation status of the sensors may be associated with the relationship between sensor pressure output and operation rate of the pump, variation in pressure (e.g., maximal pressure minus minimal pressure), and operation rate of the pump, variation in flow, etc.

As exemplified in FIGS. 7A to 7E, the pre-stored data may be processed to represent a distribution of pressure and/or flow parameters that may be measured for a given (or with respect to) operation rate of the pump, indicating the likelihood that certain pressure or flow parameters are measured for the specific operation rate of the pump. Following the collection of pressure data from the one or more sensors and determining, accordingly, one or more pressure and/or flow parameters, the processing can determine a distribution of collected pressure/flow data. Accordingly, the processing may compare the pre-stored distribution and the measured distribution, to determine a probability that the measured distribution falls within the respective pre-stored distribution (for a well operating sensor assembly). If the measured distribution falls within the pre-stored distribution, within predetermined limits, the sensor is considered as operating well. If the measured distribution is determined to be outside of the pre-stored distribution, e.g., variation between the distributions exceeds the predetermined limits, the respective one or more sensors are considered to be malfunctioning, and a proper indication is generated.

For example, given an aspiration flow rate of 60 cc/min and vacuum setpoint (pressure difference along the flow) of −700 mm/Hg, the pressure distribution is expected to be within a gaussian distribution having a mean value of −350 mm/Hg and standard deviation of 100 mmHg. In this situation, a pressure reading indicating a pressure of +50 mmHg provided by a sensor may be considered as very unlikely and indicates a likely malfunctioning sensor. While obtaining a single such reading within thousands of readout actions may be possible, repeating readings of such values may indicate a failure in the respective sensor. As a general example, when the aspiration pump 26 is operating at a selected rotation rate to build a vacuum in the aspiration path, obtaining a positive pressure readout is very unlikely, and obtaining such readouts may indicate malfunctioning sensor. Alternatively, when the irrigation pump 24 is operating at a selected rotation rate to pump irrigation fluid along the irrigation path, pressure readings along the irrigation path are expected to be positive. Obtaining a negative pressure may thus indicate a malfunctioning sensor.

### EXAMPLES

Example 1: A surgical system comprising:
(a) a handpiece (12) comprising a distal end operable for performing a surgical operation on a patient's eye (20);
(b) a pumping module comprising one or more pumps (24, 26) configured for transmitting and/or evacuating fluids via the handpiece (12);
(c) a pressure sensor assembly (51) comprising one or more pressure sensors (52a, 52b, 54, 56a, 56b, 58) positioned at one or more selected positions along a fluid flow path between the pumping module and the distal end of the handpiece (12), and configured to provide pressure data indicative of fluid pressure along the fluid flow path; and
(d) a controller (38) comprising one or more processors and memory utility (35), and configured for operating at least the pumping module;
wherein the controller (38) comprises pre-stored data, stored in the memory utility (35), the pre-stored data being indicative of a distribution of data measurable by the one or more sensors (52a, 52b, 54, 56a, 56b, 58) of fluid flow along the fluid flow path with respect to one or more rotation rate values of the one or more pumps (24, 26), and
wherein the one or more processors are configured and operable to obtain pressure data collected by the one or more sensors (52a, 52b, 54, 56a, 56b, 58), compare the pressure data to the pre-stored data and to determine accordingly an operation status of the one or more pressure sensors (52a, 52b, 54, 56a, 56b, 58).

Example 2: The surgical system of example 1, wherein the pre-stored data comprises data indicative of probability of values of the data measurable by the one or more sensors (52a, 52b, 54, 56a, 56b, 58) for a selected number of rotation rate values of the one or more pumps (24, 26).

Example 3: The surgical system of Example 1 or 2, wherein the data measurable by the one or more sensors (52, 52a, 52b, 54, 56a, 56b, 58) comprises at least one of pressure data, pressure variation, flow rate, and flow rate variation.

Example 4: The surgical system of any one of examples 1 to 3, wherein the controller (38) is operable to determine the operation status of the one or more pressure sensors (52a, 52b, 54, 56a, 56b, 58) as being operating or malfunctioning.

Example 5: The surgical system of any one of examples 1 to 4, wherein the pumping module comprises:
an irrigation module configured to supply a flow of irrigation fluid into the eye (20), the irrigation module being configured to be coupled with an irrigation channel (34a) of the handpiece (12) via an irrigation line so as to enable flow of irrigation fluid therethrough; and
an aspiration module configured for aspirating eye fluid, the aspiration module being configured to be coupled with an aspiration channel (46a) of the handpiece (12) via an aspiration line so as to enable flow of an eye fluid therethrough.

Example 6: The surgical system of example 5, wherein the sensor assembly (51) comprises:
at least one sensor (56, 58) positioned along the irrigation channel (34a) or along an irrigation line connecting the irrigation module to the irrigation channel (34a), and
at least one sensor (52, 54) positioned along the aspiration channel (46a) or along an aspiration line connecting the aspiration module to the aspiration channel (46a).

Example 7: The surgical system of any one of examples 1 to 6, wherein the sensor assembly (51) comprises two or more sensors positioned at two or more different locations along a common fluid flow path for fluid flow between the pumping module and the distal end of the handpiece (12), the controller (38) being configured and operable to receive pressure data from the two or more sensors and to determine data indicative of a flow rate of fluid along the common fluid flow path in accordance with a time delay between pressure variations detected by a first sensor and a second sensor of the two or more sensors.

Example 8: The surgical system of example 7, wherein the controller (38) is operable to determine data indicative of a flow rate of fluid along the common fluid flow path by determining a temporal correlation between a first temporal pressure profile obtained from a the first sensor, and a second temporal pressure profile obtained from the second sensor, and identifying the time delay in accordance with correlation between the first temporal pressure profile and the second temporal pressure profile.

Example 9: The surgical system of any one of examples 1 to 8, wherein the pre-stored data comprising data indicative of a probability distribution of pressure values in the fluid flow path for a selected number of rotation rate values of the one or more pumps (24, 26), and wherein the one or more processors being configured to determine a likelihood that measured data collected during a given operation rate of the respective one or more pumps (24, 26) is indicative of a faulty or operating sensor.

Example 10: The surgical system of any one of examples 1 to 9, configured as a phacoemulsification system (10), and wherein the handpiece (12) comprises a vibration generator (22), and a needle (16) and a sleeve (17) located at the distal end, the vibration generator (22) being configured to vibrate the needle (16) for emulsifying a tissue of the patient's eye (20).

Example 11: A method for sensor diagnostic in a surgical system, the method comprising:
(a) providing (4010) pre-stored data comprising a distribution of data measurable by one or more sensors along a fluid flow path transmitting fluid pumped by one or more pumps with respect to one or more rotation rate values of the one or more pumps;
(b) operating (4020) the one or more pumps (24, 26) at a selected operation rate for pumping or evacuating fluid via the fluid flow path;
(c) using (4030) the one or more pressure sensors (52, 52a, 52b, 54, 56a, 56b, 58) along the fluid flow path and providing pressure data indicative of fluid pressure at one or more locations along the fluid flow path;
(d) determining (4060) a likelihood that a measured data collected by the one or more sensor fits within the distribution in the pre-stored data, and determining accordingly an operation status of the one or more pressure sensors.

Example 12: The method of example 11, wherein the pre-stored data comprises data indicative of a distribution of the measured data on the fluid pressure and respective pump operation rate, and wherein determining a likelihood that a measured data collected by the one or more sensor fits within the distribution in the pre-stored data comprises determining a distance between the measured data and mean of the pre-stored data for a given rotation rate with respect to standard deviation of the pre-stored data.

Example 13: The method of example 11 or 12, wherein using one or more pressure sensors comprises using two or more pressure sensors located at two or more different locations along the fluid flow path, the method further comprising determining (5070) a fluid flow rate in the fluid flow path in accordance with a temporal correlation between pressure data measured by the two or more pressure sensors, and respective locations thereof.

Example 14: The method of any one of examples 11 to 13, wherein the pre-stored data comprises data indicative of a typical behavior of data on fluid pressure measurable by the one or more sensors and the rotation rate values of the one or more pumps, the pre-stored data comprises data indicative of probability of values of the data measurable by the two or more sensors for different values of rotation rate of the one or more pumps.

Example 15: The method of any one of examples 11 to 14, wherein the operation status of the one or more pressure sensors is determined as being operating or malfunctioning.

Example 16: a surgical system comprising:
(e) a handpiece (12) comprising a distal end operable for performing a surgical operation on a patient's eye (20);
(f) a pumping module comprising one or more pumps (24, 26) configured for transmitting and/or evacuating fluids via the handpiece (12);
(g) a pressure sensor assembly (51) comprising one or more pressure sensors (52a, 52b, 54, 56a, 56b, 58) positioned at one or more selected positions along a fluid flow path between the pumping module and the distal end of the handpiece (12), and configured to provide pressure data indicative of fluid pressure along the fluid flow path; and
(h) a controller (38) comprising one or more processors and memory utility (35), and configured for operating at least the pumping module;
wherein the controller (38) comprises pre-stored data, stored in the memory utility (35), the pre-stored data being indicative of a distribution of data measurable by the one or more sensors (52a, 52b, 54, 56a, 56b, 58) of fluid flow along the fluid flow path with respect to one or more rotation rate values of the one or more pumps (24, 26), and
wherein the one or more processors are configured and operable to obtain pressure data collected by the one or more sensors (52a, 52b, 54, 56a, 56b, 58), compare the pressure data to the pre-stored data and to determine accordingly an operation status of the one or more pressure sensors (52a, 52b, 54, 56a, 56b, 58).

Example 17: The surgical system of example 16, wherein the pre-stored data comprises data indicative of probability of values of the data measurable by the one or more sensors (52a, 52b, 54, 56a, 56b, 58) for a selected number of rotation rate values of the one or more pumps (24, 26).

Example 18: The surgical system of Example 16, wherein the data measurable by the one or more sensors (52, 52a, 52b, 54, 56a, 56b, 58) comprises at least one of pressure data, pressure variation, flow rate, and flow rate variation.

Example 19: The surgical system of any one of examples 16, wherein the controller (38) is operable to determine the operation status of the one or more pressure sensors (52a, 52b, 54, 56a, 56b, 58) as being operating or malfunctioning.

Example 20: The surgical system of any one of examples 16, wherein the pumping module comprises:
an irrigation module configured to supply a flow of irrigation fluid into the eye (20), the irrigation module being configured to be coupled with an irrigation channel (34a) of the handpiece (12) via an irrigation line so as to enable flow of irrigation fluid therethrough; and
an aspiration module configured for aspirating eye fluid, the aspiration module being configured to be coupled with an aspiration channel (46a) of the handpiece (12) via an aspiration line so as to enable flow of an eye fluid therethrough; and
wherein the sensor assembly (51) comprises:
at least one sensor (56, 58) positioned along the irrigation channel (34a) or along an irrigation line connecting the irrigation module to the irrigation channel (34a), and
at least one sensor (52, 54) positioned along the aspiration channel (46a) or along an aspiration line connecting the aspiration module to the aspiration channel (46a).

Example 21: The surgical system of any one of examples 16 to 19, wherein the sensor assembly (51) comprises two or more sensors positioned at two or more different locations along a common fluid flow path for fluid flow between the pumping module and the distal end of the handpiece (12), the controller (38) being configured and operable to receive pressure data from the two or more sensors and to determine data indicative of a flow rate of fluid along the common fluid flow path in accordance with a time delay between pressure variations detected by a first sensor and a second sensor of the two or more sensors.

Example 22: The surgical system of example 21, wherein the controller (38) is operable to determine data indicative of a flow rate of fluid along the common fluid flow path by determining a temporal correlation between a first temporal pressure profile obtained from the first sensor, and a second temporal pressure profile obtained from the second sensor, and identifying the time delay in accordance with correlation between the first temporal pressure profile and the second temporal pressure profile.

Example 23: A method for sensor diagnostic in a surgical system, the method comprising:
(e) providing (4010) pre-stored data comprising a distribution of data measurable by one or more sensors along a fluid flow path transmitting fluid pumped by one or more pumps with respect to one or more rotation rate values of the one or more pumps;
(f) operating (4020) the one or more pumps (24, 26) at a selected operation rate for pumping or evacuating fluid via the fluid flow path;
(g) using (4030) the one or more pressure sensors (52, 52a, 52b, 54, 56a, 56b, 58) along the fluid flow path and providing pressure data indicative of fluid pressure at one or more locations along the fluid flow path;
(h) determining (4060) a likelihood that a measured data collected by the one or more sensor fits within the distribution in the pre-stored data, and determining accordingly an operation status of the one or more pressure sensors.

Example 24: The method of example 23, wherein using one or more pressure sensors comprises using two or more pressure sensors located at two or more different locations along the fluid flow path, the method further comprising determining (5070) a fluid flow rate in the fluid flow path in accordance with a temporal correlation between pressure data measured by the two or more pressure sensors, and respective locations thereof.

Example 25: The method of any one of examples 23 or 24, wherein the pre-stored data comprises data indicative of a typical behavior of data on fluid pressure measurable by the one or more sensors and the rotation rate values of the one or more pumps, the pre-stored data comprises data indicative of probability of values of the data measurable by the two or more sensors for different values of rotation rate of the one or more pumps.

The present disclosure thus provides a surgical system, e.g., a phacoemulsification system, and a respective method enabling monitoring of the operation status of one or more pressure sensors. The present technique provides monitoring of sensor conditions in accordance with pre-stored data indicative of sensor operation, enabling generation of an alert when one or more sensors malfunction, while allowing a physician to proceed with a surgical operation, and reducing the risk of damage to the patient's eye. The technique of the present disclosure utilizes a pressure sensor assembly comprising one or more sensors and processing of correlation of pressure readings for a given operation rate of a respective pump, with respect to pre-stored historical data on pressure and/or flow parameters. The technique of the present disclosure may also operate to update the pre-stored data and may also utilize correlation between pressure readings of the sensors and rotation rate of the respective pump.

It is to be noted that the various features described in the various examples can be combined according to all possible technical combinations.

It is to be understood that the invention is not limited in its application to the details set forth in the description contained herein or illustrated in the drawings. The invention is capable of other examples and of being practiced and carried out in various ways. Hence, it is to be understood that the phraseology and terminology employed herein are for the purpose of description and should not be regarded as limiting. As such, those skilled in the art will appreciate that the conception upon which this disclosure is based can readily be utilized as a basis for designing other structures, methods, and systems for carrying out the several purposes of the presently disclosed subject matter.

Those skilled in the art will readily appreciate that various modifications and changes can be applied to the examples of the invention as hereinbefore described without departing from its scope, defined in and by the appended claims.

1. A surgical system comprising:
(a) a handpiece comprising a distal end operable for performing a surgical operation on a patient's eye;
(b) a pumping module comprising one or more pumps configured for transmitting and/or evacuating fluids via the handpiece;
(c) a pressure sensor assembly comprising one or more pressure sensors positioned at one or more selected positions along a fluid flow path between the pumping module and the distal end of the handpiece, and configured to provide pressure data indicative of fluid pressure along the fluid flow path; and
(d) a controller comprising one or more processors and memory utility, and configured for operating at least the pumping module;
wherein the controller comprises pre-stored data, stored in the memory utility, the pre-stored data being indicative of a distribution of data measurable by the one or more sensors of fluid flow along the fluid flow path with respect to one or more rotation rate values of the one or more pumps, and
wherein the one or more processors are configured and operable to obtain pressure data collected by the one or more sensors, compare the pressure data to the pre-stored data and to determine accordingly an operation status of the one or more pressure sensors.

2. The surgical system of claim 1, wherein the pre-stored data comprises data indicative of probability of values of the data measurable by the one or more sensors for a selected number of rotation rate values of the one or more pumps.

3. The surgical system of claim 1, wherein the data measurable by the one or more sensors comprises at least one of pressure data, pressure variation, flow rate, and flow rate variation.

4. The surgical system of claim 1, wherein the controller is operable to determine the operation status of the one or more pressure sensors as being operating or malfunctioning.

5. The surgical system of claim 1, wherein the pumping module comprises:
an irrigation module configured to supply a flow of irrigation fluid into the eye, the irrigation module being configured to be coupled with an irrigation channel of the handpiece via an irrigation line so as to enable flow of irrigation fluid therethrough; and
an aspiration module configured for aspirating eye fluid, the aspiration module being configured to be coupled with an aspiration channel of the handpiece via an aspiration line so as to enable flow of an eye fluid therethrough.

6. The surgical system of claim 5, wherein the sensor assembly comprises:
at least one sensor positioned along the irrigation channel or along an irrigation line connecting the irrigation module to the irrigation channel, and
at least one sensor positioned along the aspiration channel or along an aspiration line connecting the aspiration module to the aspiration channel.

7. The surgical system of claim 1, wherein the sensor assembly comprises two or more sensors positioned at two or more different locations along a common fluid flow path between the pumping module and the distal end of the handpiece, the controller being configured and operable to receive pressure data from the two or more sensors and to determine data indicative of a flow rate of fluid along the common fluid flow path in accordance with a time delay between pressure variations detected by a first sensor and a second sensor of the two or more sensors.

8. The surgical system of claim 7, wherein the controller is operable to determine data indicative of a flow rate of fluid along the common fluid flow path by determining a temporal correlation between a first temporal pressure profile obtained from the first sensor, and a second temporal pressure profile obtained from the second sensor, and identifying the time delay in accordance with correlation between the first temporal pressure profile and the second temporal pressure profile.

9. The surgical system of claim 1, wherein the pre-stored data comprising data indicative of a probability distribution of pressure values in the fluid flow path for a selected number of rotation rate values of the one or more pumps, and wherein the one or more processors being configured to determine a likelihood that measured data collected during a given operation rate of the respective one or more pumps is indicative of a faulty or operating sensor.

10. The surgical system of claim 1, wherein the handpiece comprises a vibration generator, and a needle and a sleeve located at the distal end, the vibration generator being configured to vibrate the needle for emulsifying a tissue of the patient's eye.

11. The surgical system of claim 1, configured as a phacoemulsification system.

12. A phacoemulsification system comprising:
(a) a handpiece comprising a piezoelectric element and a distal end comprising a needle and a sleeve, wherein the distal end is configured to be inserted into an eye and to be vibrated by the piezoelectric element to emulsify a lens of the eye;
(b) an irrigation module configured to supply a flow of irrigation fluid into the eye, the irrigation module comprising at least one pump and configured to be coupled with an irrigation channel of the handpiece via an irrigation line so as to transmit an irrigation fluid therethrough;
(c) an aspiration module configured for aspirating eye fluid, the aspiration module comprising at least one pump and configured to be coupled with an aspiration channel of the handpiece via an aspiration line so as to evacuate fluid and tissue material from the eye;
(d) a pressure sensor assembly comprising one or more pressure sensors coupled with at least one of the irrigation channel, the irrigation line, the aspiration channel, and the aspiration line, and configured to monitor at least one of an irrigation pressure in the irrigation channel, an irrigation pressure in the irrigation line, an aspiration pressure in the aspiration channel, and an aspiration pressure in the aspiration line, and to generate pressure data indicative of fluid pressure; and
(e) a controller comprising one or more processors memory utility and configured for operating the irrigation module and aspiration module;
wherein the controller comprises pre-stored data, stored in the memory utility, the pre-stored data being indicative of a distribution of data measurable by the one or more sensors along a fluid flow path with respect to one or more rotation rate values of the one or more pumps, and
wherein the one or more processors are configured and operable to obtain pressure data collected by the one or more sensors, compare the pressure data to the pre-stored data and to determine accordingly an operation status of the one or more pressure sensors.

13. A method for sensor diagnostic in a surgical system, the method comprising:
(a) providing pre-stored data comprising a distribution of data measurable by one or more sensors along a fluid flow path with respect to one or more rotation rate values of one or more pumps;
(b) operating the one or more pumps at a selected operation rate for pumping or evacuating fluid via the fluid flow path;
(c) using the one or more pressure sensors along the fluid flow path and providing pressure data indicative of fluid pressure at one or more locations along the fluid flow path;
(d) determining a likelihood that a measured data collected by the one or more sensor fits within the distribution in the pre-stored data, and determining accordingly an operation status of the one or more pressure sensors.

14. The method of claim 13, further comprising processing the pressure data provided by two or more sensors to determine the measured data on the fluid pressure, the measured data on fluid pressure comprises one or more of pressure data and flow parameters of fluid along the fluid flow path.

15. The method of claim 13, wherein the pre-stored data comprises data indicative of a distribution of the measured data on the fluid pressure and respective pump operation rate, and wherein determining a likelihood that a measured data collected by the one or more sensor fits within the distribution in the pre-stored data comprises determining a distance between the measured data and mean of the pre-stored data for a given rotation rate with respect to standard deviation of the pre-stored data.

16. The method of claim 13, wherein using one or more pressure sensors comprises using two or more pressure sensors located at two or more different locations along the fluid flow path, the method further comprising determining a fluid flow rate in the fluid flow path in accordance with a temporal correlation between pressure data measured by the two or more pressure sensors, and respective locations thereof.

17. The method of claim 13, wherein the operation status of the one or more pressure sensors is determined as being operating or malfunctioning.

18. The method of claim 13, wherein using one or more pressure sensors comprises using two or more pressure sensors positioned at two different locations along the fluid flow path, and providing pressure data comprising determining data indicative of a flow rate of fluid along the fluid flow path in accordance with a time delay between pressure variations detected by a first sensor and a second sensor of the two or more sensors.

19. The method of claim 18, wherein determining data indicative of a flow rate of fluid along the fluid flow path comprises determining a temporal correlation between a first temporal pressure profile obtained from the first sensor, and a second temporal pressure profile obtained from the second sensor, and identifying the time delay in accordance with a correlation between the first temporal pressure profile and the second temporal pressure profile.

20. The method of claim 13, wherein the pre-stored data comprises data indicative of a typical behavior of data on fluid pressure measurable by the one or more sensors and the rotation rate values of the one or more pumps, the pre-stored data comprises data indicative of probability of values of the data measurable by the two or more sensors for different values of rotation rate of the one or more pumps.
